(19) United States
(12) Patent Application Publication
(10) Pub. No.: US 2013/0261746 A1
(43) Pub. Date: Oct. 3, 2013

(54) IMPLANTABLE INTER-VERTEBRAL DISK HAVING UPPER AND LOWER LAYERS OF A METAL EXHIBITING BONE FUSING CHARACTERISTICS AND WHICH SANDWICH THEREBETWEEN A SOFT PLASTIC CUSHIONING DISC FOR PROVIDING DYNAMIC PROPERTIES MIMICKING THAT OF A NATURAL INTER-VERTEBRAL DISC

(71) Applicant: LINARES MEDICAL DEVICES, LLC, Auburn Hills, MI (US)

(72) Inventors: Miguel A. Linares, Bloomfield Hills, MI (US); Ryan T. Greene, Bloomfield Hills, MI (US); Miguel A. Linares, JR., Bloomfield Hills, MI (US)

(73) Assignee: Linares Medical Devices, LLC, Auburn Hills, MI (US)

(21) Appl. No.: 13/840,701

(22) Filed: Mar. 15, 2013

Related U.S. Application Data
(60) Provisional application No. 61/616,651, filed on Mar. 28, 2012.

(51) Int. Cl. A61F 2/44

(52) U.S. Cl. CPC A61F 2/442
USPC 623/17.16

(57) ABSTRACT

The present invention discloses an inter-vertebral and implantable disk substituting for a fibro cartilage and including first and second hard outer layers between which is sandwiched a softer inner cushioning layer. Each of the outer layers includes an exterior texturized pattern for promoting bone in-growth subsequent to implantation. Each of the outer layers may also include an exteriorly integrally formed and angled mounting portion including an interiorly positioned aperture. The mounting portions are adapted to being applied against reconditioned side exterior locations of first and second succeeding intervertebral bodies and mounted to the bodies with a screw or clip.

### CROSS-REFERENCE TO RELATED APPLICATIONS

This Application claims the benefit of U.S. Provisional Application 61/616,651 filed on Mar. 28, 2012, the contents of which are incorporated herein in its entirety.

### FIELD OF THE INVENTION

The present invention relates generally to an implantable inter-vertebral disk for placement between opposing body portions of succeeding vertebra, such as associated with but not limited to a human spine. More specifically, the present invention disclose an artificial disk which replaces a previously existing and damaged or otherwise degraded inter-vertebral fibro cartilage, the artificial disk exhibiting outer layers of metal exhibiting bone fusing characteristics which sandwich therebetween a soft plastic cushioning layer which, upon being implanted, mimics the characteristics of natural fibro cartilage.

### BACKGROUND OF THE INVENTION

The prior art is documented with examples of inter-vertebral body implants, such as disks or cushions. A first example of this is the flanged interbody spinal fusion implant of Michelson, U.S. Pat. No. 6,730,127. The flange body exhibits bone screw receiving holes adapted to overly each of the adjacent vertebral bodies. The implant has at least one locking element to lock one or more of the bone screws inserted into the receiving holes.

McLeod, US 2009/0105826 teaches disc prosthesis for use in the lumbar spine region and including a core consisting of one or more filling elements provided within an inner component of fabric. The inner component is provided within an outer component of fabric. A smooth inner contacting surface between the inner component and the core filling facilitates movement between the inner component and the core.

Seifert 2011/0251689 teaches an intervertebral implant in which a spacer portion includes inferior and superior surfaces, each of which have a contact area capable of engaging with the anatomy in the treated intervertebral space. A through hole extends through the spacer body. Screw holes extend from a side portion to the inferior and superior surfaces of the spacer portion and plate portion containing screw receiving holes is rigidly coupled to the spacer portion through a coupling means. A screw back out prevention mechanism on the plate portion prevents back out of screws from the holes and to secure the spacer portion to the plat portion of the implant.

Finally, US 2011/0106262 teaches a prosthetic intervertebral disc and method in which upper and lower endplates are separated by a compressible core member. A series of one, two, three and four piece structures are provided and exhibit stiffness in the vertical direction, torsional stiffness, bending stiffness in the sagittal plane, and bending stiffness in the front plane, and in which the degree of these features can be controlled independently by adjusting the components of the discs. An interface mechanism between the endplates and the core members across the several embodiments facilitates surgical implantation.

### SUMMARY OF THE INVENTION

The present invention discloses an inter-vertebral and implantable disk substituting for a fibro cartilage and including first and second hard outer layers between which is sandwiched a softer inner cushioning layer. Each of the outer layers includes an exterior texturized pattern for promoting bone in-growth subsequent to implantation.

Each of the outer layers may also include an exteriorly integrally formed and angled mounting portion including an interiorly positioned aperture. The mounting portions are adapted to being applied against reconditioned side exterior locations of first and second succeeding intervertebral bodies and mounted to the bodies with a screw or clip.

Other features include the outer layers being constructed of any of titanium, medical grade stainless steel, other metal, composite metal/polymer, or composite polymer material. The inner cushioning layer may also include a material with a lower/softer durometer rating less than that associated with a harder/higher rating of the hard outer layers.

An inner surface associated with each of the hard outer layers may also integrate an arrangement of keyed inward projections in a staggered or offset fashion relative to the inner sandwiched cushioning layer to provide better bond line maintenance along boundary interfaces established between the inner surfaces and the cushioning layer and to further provide a degree of anti-shear support relative to the boundary interfaces in order to prevent inadvertent separation of the cushioning material from either of the hard outer layers.

Other features include the exterior texturized pattern exhibiting a plurality of laser cut and intersecting grid lines establishing an angle relative to a perpendicular established with a face of the layer. The exterior texturized pattern may also incorporate any of a repetitive wave pattern combined with iteratively located apertures for further promoting bone in-growth, multiple spiral patterns, or an irregular and cavitation surface pattern.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is a perspective illustration of the inter-vertebral disk implant installed between opposing faces of first and second vertebral bodies;

FIG. 2 is a rotated perspective similar to FIG. 1 with a given vertebral body removed and in order to better illustrate the texturized exterior associated with a selected hard outer layer for encouraging bone ingrowth and fusion upon implantation;

FIG. 3 is a rotated and linear cutaway perspective of the assembly shown in FIG. 1 and better depicting the interlocking and sandwiching profile established between the hard outer layers and the inner softer cushioning layer, this including keyed inward projections associated with the outer layers in order to provide better bond line maintenance and anti-shear support relative to the boundary interfaces established with the inner sandwiched cushioning material;

FIG. 4 is an enlarged partial perspective of a laser pattern formed on the exterior of each hard outer layer, such as in order to establish the texturized pattern depicted in FIG. 2, the laser pattern including one non-limiting example of angled and grid-like intersecting lines in order to enhance in-growth and bonding of bone;

FIG. 5 is an illustration similar to FIG. 4 of a further texturized pattern in the form of an electro-discharge (EDM) or spark erosion machining process using a numerically controlled carbon tungsten or like tool for creating a repetitive wave pattern combined with iteratively located apertures for further promoting bone in-growth;

FIG. 6 is a further alternate illustration to either of FIGS. 4 and 5 and which depicts multiple spiral patterns formed into the surface of the hard outer wear layer in order to promote bone in-growth; and

FIG. 7 is a yet further alternate illustration to any of FIGS. 4-6 and which depicts a grit blasted texture associated with the hard outer wear layer for promoting bone fusion.

### DETAILED DESCRIPTION OF THE PREFERRED EMBODIMENTS

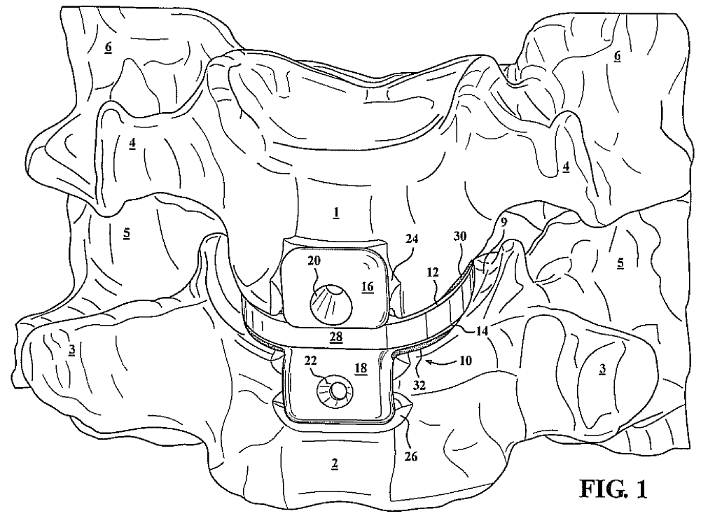

As previously described, the present invention disclose an artificial disk assembly, which is generally depicted at 10 in each of FIGS. 1-3, and such as which is designed to replace a previously existing and damaged or otherwise degraded inter-vertebral fibro cartilage (not shown). As is known, a naturally occurring inter-vertebral disk (or inter-vertebral cartilage) is constructed of an outer annulus fibrosis which consists of several layers of fibro-cartilage, this surrounding an inner nucleus pulposus which contains loose fibers suspended in a mucoprotein gel with a consistency not unlike jelly.

In this manner, the nucleus of the disk acts as a shock absorber in order to absorb the impact of the body's daily activities along with keeping the opposing vertebrae separated. As further depicted in each of FIGS. 1-3, and as best shown in FIG. 1, a first upper disk includes a main body 1 and a second lower disk a main body 2, between which the implant disk assembly 10 is installed.

Without embarking upon a detailed physiological description of the human anatomy, suffice it to say that the human spine includes twenty three disks, including six in the neck (cervical) region, twelve in the middle back (thoracic) region, and an additional five in the lower back (lumbar) region. As again shown in FIG. 1, first and second vertebrae are representative of either of the lumbar or thoracic region, such that each includes a pair of superior articular processes (3 for lower vertebrae 2 and 4 for upper vertebrae 1), as well as a further pair of transverse processes (5 for lower vertebrae 2 and 6 for upper vertebrae 1). Largely hidden from view are additional facets or portions including the inferior articular process, the foramen transversium, and the like.

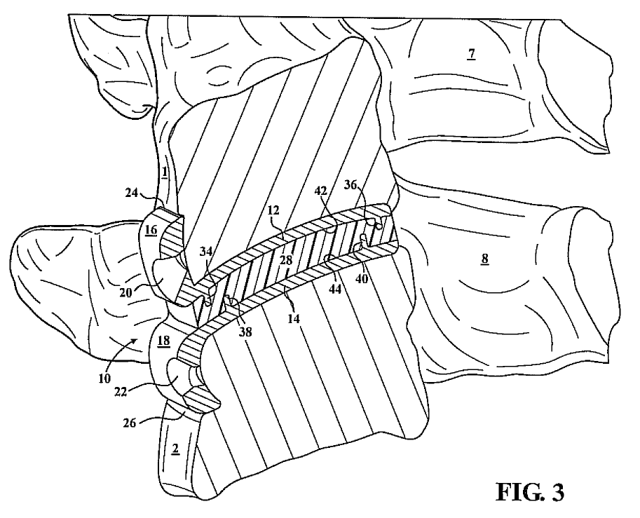

Rear spinous processes are further shown in the rotated and partially sectioned view of FIG. 3 and include such at that shown at 7 for upper vertebrae 1 and 8 for lower vertebrae 2. A pedicle 9 is further best depicted in FIG. 1 in relation to the lower positioned vertebrae and which connects the forward body 2 with the various superior 3, transverse 5 and spinous 8 processes as well as defining part of a rearwardly located and hidden open interior defined vertebral foramen.

For purposes of ease of illustration, also not depicted are a collection of ligament structures surrounding the spinal column, these including such as the anterior longitudinal ligaments which overlay the bodies 1 and 2 and inter-disposed disk assembly 10 (this again in substitution of the inter-vertebral fibro-cartilage), the posterior longitudinal ligaments (this located on the opposite rear side of the bodies 1 and 2 proximate the interconnecting pedicle 9 and the vertebral foramen, the capsulary ligaments extending between the superior articular facets or processes 3 and 4, as well as related inter-vertebral fibro cartilage and anterior costo-transverse ligaments, the inter-spinal ligaments associated with the spinous processes 7 and 8, and the supra-spinal ligaments extending from the rear of the column. Also not shown is the spinal nerve cord and individual branches associated with the column and which is outside of the scope of this description.

Having undertaken a general description of the associated anatomy with which the present invention cooperates, the artificial disk 10 as generally depicted exhibits a pair of outer layers 12 and 14 of a hard material, such as including but not limited to any of a titanium, other medical grade stainless steel, other metal or any hard composite material not limited to plastic/metal or various polymeric compositions exhibiting a hard outer consistency including such as medical grade nylon or other material. As shown collectively in FIGS. 1-3, each of the upper layer 12 and lower spaced apart layer 14 also include an exteriorly integrally formed and angled mounting portion or angled end bracket (at 16 for upper layer 12 and at 18 for lower layer 14).

Each of the angled end brackets includes one or more interiorly positioned apertures, such as depicted by closed perimeter defining surface 20 for angled end bracket 16 associated with layer 12, further by closed perimeter surface 22 for angled end bracket 18 associated with layer 14. One or more screws, clips or other suitable fastener, such as generally depicted at 21 and 23, can be provided and in order to mount to reconditioned side disposed and exterior facing locations 24 and 26 respectively of the vertebral bodies 1 and 2.

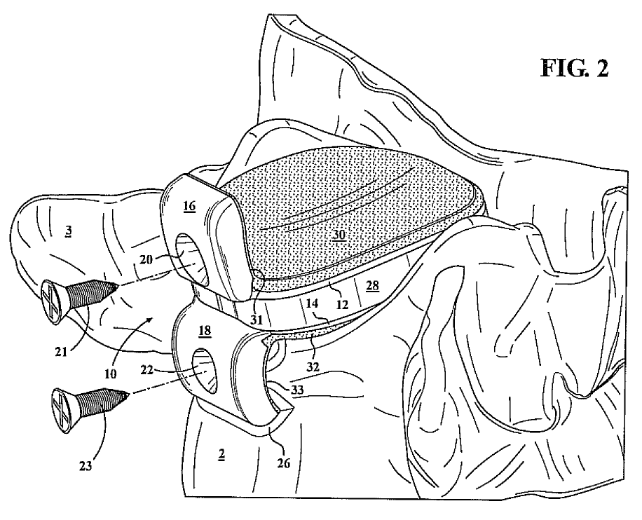

Sandwiched between the hard outer layers 12 and 14 is a soft plastic cushioning layer 28 (such as including any lower durometer rated polymer or like material) and which, upon being implanted, mimics the characteristics of natural inner nucleus pulposus (such as including exhibiting an equivalent durometer rating as associated with the original cartilage material), with the hard outer layers 12 and 14 likewise mimicking that of natural fibro cartilage. Each of FIGS. 1 and 2 further depict a texturized exterior, at 30 and 32 associated with hard outer layers 12 and 14 respectively and as best shown in FIG. 2 by the texturized surface 30 associated with upper layer 12 for encouraging bone ingrowth and fusion subsequent to implantation between the opposing end faces of the vertebral bodies 1 and 2 (and again such as following previous surgical removal of damaged or insufficient fibrocartilage.

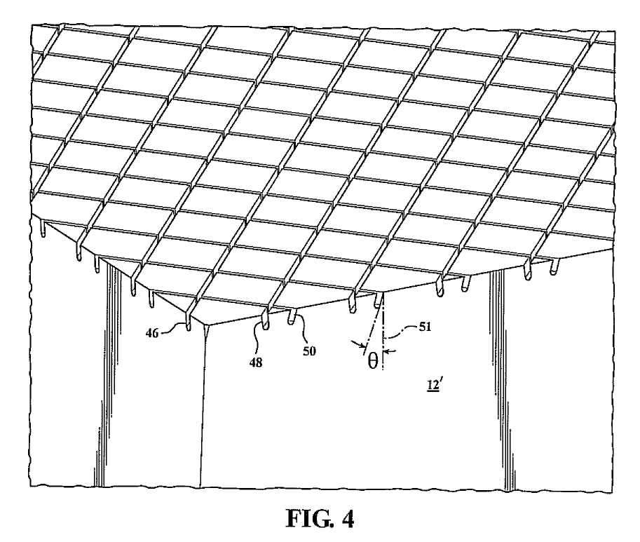

FIG. 2 also best depicts the manner in which the textured surface additionally extends (as shown at 31 extending from surface 30 and further at 33 from surface 32) over the underside of each of the angled end brackets 16 and 18. Multiple examples of other effective texturizing patterns and consistencies will be had upon reference to those set forth in non-limiting fashion in each of succeeding FIGS. 4-7.

FIG. 3 is a rotated and linear cutaway perspective of the assembly shown in FIG. 1 and better depicting the interlocking and sandwiching profile established between the hard outer layers 12 and 14, and the inner softer cushioning layer 28. As shown, this includes the provision of keyed inward projections (see at 34 and 36 with upper layer 12 and further at 38 and 40 with lower layer 14).

The keyed projections 34 & 36 and 38 & 40 are depicted in a staggered or offset fashion relative to the inner sandwiched cushioning layer 28, such as in order to provide better bond line maintenance along boundary interfaces 42 and 44 established between the inner surfaces of the outer layers 12 and 14 and the intermediate cushioning layer 28, this further providing a degree of anti-shear support relative to the boundary interfaces 42 and 44 and in order to prevent inadvertent separation of the cushioning material 28 from either of the hardened upper 12 and lower 14 layers.

Referring now to FIG. 4, an enlarged partial perspective is generally depicted of pluralities of intersecting laser patterns 46, 48, 50, et seq., which are formed on the exterior of each hard outer layer (generally represented by modified upper layer 12′) and such as in order to establish the texturized pattern equivalently depicted in FIG. 2. The laser pattern as depicted includes one non-limiting example of angled (see angle □ shown) profiles relative to a perpendicular axis, at 51, established with a flat surface of the pattern. The grid-like profile is exhibited by pluralities of intersecting lines, this in order to enhance in-growth and bonding of bone additional by providing additional bond line geometry beyond what may be obtainable from linear or perpendicular extending laser cut lines.

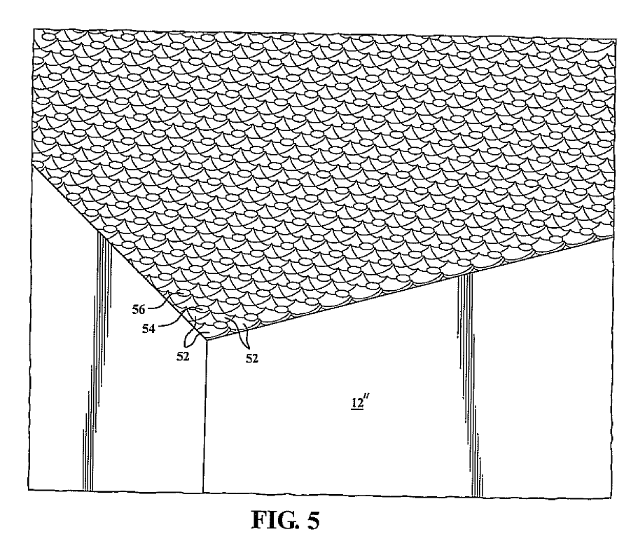

FIG. 5 is an illustration similar to FIG. 4 of a further texturized pattern formed in a further example of a modified outer layer 12″ in the form of an electro-discharge (EDM) or spark erosion machining process, such as in one non-limiting process application utilizing a numerically controlled carbon tungsten or like tool for creating a repetitive wave pattern, see as at depicted at 52 by multiple interconnecting and individual concave shaped locations which define peaks and valleys, this being combined with iteratively located apertures 52, 54, 56 which are either drilled or EDM spark eroded into the layer 12″ for further promoting additional bone in-growth.

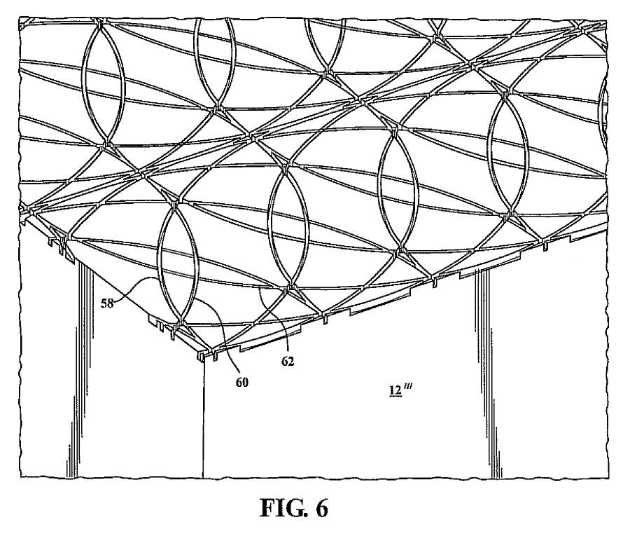

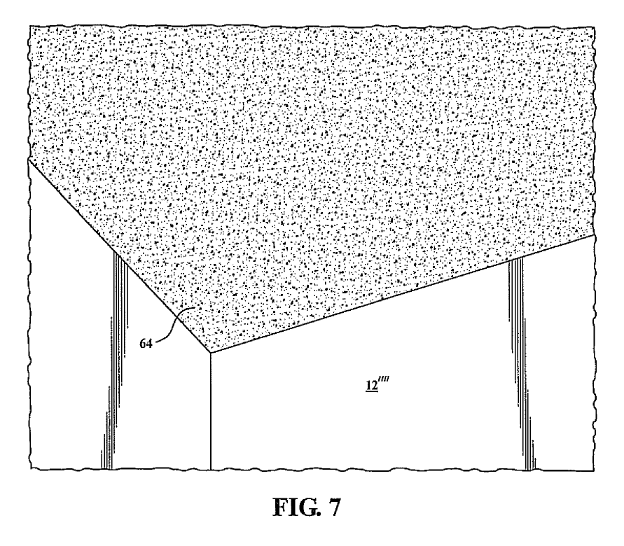

FIG. 6 is a further alternate illustration to either of FIGS. 4 and 5 and which depicts multiple spiral patterns 58, 60, 62, et seq. formed into the surface of a further version 12′″ of a hard outer wear layer in order to promote bone in-growth. Finally, FIG. 7 is a yet further alternate illustration to any of FIGS. 4-6 and which depicts a grit blasted texture 64 associated with a yet further variation of hard outer wear layer 12″″ for promoting bone fusion. For purposes of description, any grade or variety of grit can be substituted or mixed in order to achieve any desired irregular or cavitation pattern for optimizing bone fusion.

Having described my invention, other and additional preferred embodiments will become apparent to those skilled in the art to which it pertains, and without deviating from the scope of the appended claims.

We claim:

1. An inter-vertebral and implantable disk substituting for a fibro cartilage, comprising:
first and second hard outer layers between which is sandwiched a softer inner cushioning layer; and
each of said outer layers including an exterior texturized pattern for promoting bone in-growth subsequent to implantation.

2. The implantable disk as described in claim 1, each of said outer layers further comprising an exteriorly integrally formed and angled mounting portion including an interiorly positioned aperture, said mounting portions adapted to being applied against reconditioned side exterior locations of first and second succeeding intervertebral bodies and mounted to the bodies with a screw or clip.

3. The implantable disk as described in claim 1, said outer layers each further comprising any of titanium, medical grade stainless steel, other metal, composite metal/polymer, or composite polymer material.

4. The implantable disk as described in claim 1, said inner cushioning layer further comprising a material with a lower/softer durometer rating less than that associated with a harder/higher rating of said hard outer layers.

5. The implantable disk as described in claim 1, an inner surface associated with each of said hard outer layers further comprising an arrangement of keyed inward projections in a staggered or offset fashion relative to said inner sandwiched cushioning layer to provide better bond line maintenance along boundary interfaces established between said inner surfaces and said cushioning layer and further providing a degree of anti-shear support relative to the boundary interfaces in order to prevent inadvertent separation of said cushioning material from either of said hard outer layers.

6. The implantable disk as described in claim 1, said exterior texturized pattern further comprising a plurality of laser cut and intersecting grid lines establishing an angle relative to a perpendicular established with a face of said layer.

7. The implantable disk as described in claim 1, said exterior texturized pattern further comprising a repetitive wave pattern combined with iteratively located apertures for further promoting bone in-growth.

8. The implantable disk as described in claim 1, said exterior texturized pattern further comprising multiple spiral patterns.

9. The implantable disk as described in claim 1, said exterior texturized pattern further comprising an irregular and cavitation surface pattern.

10. An inter-vertebral and implantable disk substituting for a fibro cartilage, comprising:
first and second hard outer layers between which is sandwiched a softer inner cushioning layer;
each of said outer layers including an exterior texturized pattern for promoting bone in-growth subsequent to implantation; and
each of said outer layers further comprising an exteriorly integrally formed and angled mounting portion including an interiorly positioned aperture, said mounting portions adapted to being applied against reconditioned side exterior locations of first and second succeeding intervertebral bodies and mounted to the bodies with a screw or clip.

11. The implantable disk as described in claim 10, said outer layers each further comprising any of titanium, medical grade stainless steel, other metal, composite metal/polymer, or composite polymer material.

12. The implantable disk as described in claim 10, said inner cushioning layer further comprising a material with a lower/softer durometer rating less than that associated with a harder/higher rating of said hard outer layers.

13. The implantable disk as described in claim 10, an inner surface associated with each of said hard outer layers further comprising an arrangement of keyed inward projections in a staggered or offset fashion relative to said inner sandwiched cushioning layer to provide better bond line maintenance along boundary interfaces established between said inner surfaces and said cushioning layer and further providing a degree of anti-shear support relative to the boundary interfaces in order to prevent inadvertent separation of said cushioning material from either of said hard outer layers.

14. The implantable disk as described in claim 10, said exterior texturized pattern further comprising a plurality of laser cut and intersecting grid lines establishing an angle relative to a perpendicular established with a face of said layer.

15. The implantable disk as described in claim 10, said exterior texturized pattern further comprising a repetitive wave pattern combined with iteratively located apertures for further promoting bone in-growth.

16. The implantable disk as described in claim 10, said exterior texturized pattern further comprising multiple spiral patterns.

17. The implantable disk as described in claim 10, said exterior texturized pattern further comprising an irregular and cavitation surface pattern.

18. An inter-vertebral and implantable disk substituting for a fibro cartilage, comprising:
first and second hard outer layers between which is sandwiched a softer inner cushioning layer, said outer layers each further comprising any of titanium, medical grade stainless steel, other metal, composite metal/polymer, or composite polymer material; said inner cushioning layer further comprising a material with a lower/softer durometer rating less than that associated with a harder/higher rating of said hard outer layers; and
each of said outer layers including an exterior texturized pattern for promoting bone in-growth subsequent to implantation.

19. The implantable disk as described in claim 18, each of said outer layers further comprising an exteriorly integrally formed and angled mounting portion including an interiorly positioned aperture, said mounting portions adapted to being applied against reconditioned side exterior locations of first and second succeeding intervertebral bodies and mounted to the bodies with a screw or clip.

20. The implantable disk as described in claim 18, each of said outer layers further comprising an exteriorly integrally formed and angled mounting portion including an interiorly positioned aperture, said mounting portions adapted to being applied against reconditioned side exterior locations of first and second succeeding intervertebral bodies and mounted to the bodies with a screw or clip.
